(19) United States
(12) Patent Application Publication
(10) Pub. No.: US 2014/0058430 A1
(43) Pub. Date: Feb. 27, 2014

(54) INTRANASAL BONE REMODELING DEVICES AND THEIR PLACE IN MINIMALLY INVASIVE SINUS PROCEDURES

(71) Applicant: Leonard V. Covello, Munster, IN (US)

(72) Inventor: Leonard V. Covello, Munster, IN (US)

(21) Appl. No.: 13/903,583

(22) Filed: May 28, 2013

Related U.S. Application Data
(63) Continuation-in-part of application No. 12/804,398, filed on Jul. 20, 2010.
(60) Provisional application No. 61/652,626, filed on May 29, 2012.

(51) Int. Cl. A61B 17/34

(52) U.S. Cl. CPC A61B 17/3472
USPC 606/185

(57) ABSTRACT

A method is provided for accessing and treating at least one of a maxillary sinusitis, sphenoid sinusitis and frontal sinusitis. A perforation is created in an uncinate via an anterior keyhole. A position of an MSO is verified using a probe. The probe is used to verify dimensions of the MSO. A verification is made that an anteroposterior length of the ostium is sufficient. A targeted medial displacement of a medial wall of the maxillary sinus and uncinate is made using an uncinate medializing device.

### CROSS-REFERENCE TO RELATED APPLICATIONS

This application claims the benefit of U.S. 61/652,626 filed May 29, 2012 and is a continuation-in-part of U.S. Ser. No. 12/804,398 filed Jul. 20, 2010, both of which applications are fully incorporated herein by reference.

### BACKGROUND OF THE INVENTION

1. Field of the Invention

The present invention relates generally to minimally invasive devices, systems and methods for accessing and treating maxillary sinusitis, sphenoid sinusitis, and frontal sinusitis, and more particularly for intranasal bone remodeling devices and methods for minimally invasive devices and methods for treating maxillary, sphenoid, and frontal sinusitis.

2. Description of the Related Art

There are a substantial number of people with sinus inflammatory disease—sinusitis—that could benefit from sinus surgery. Patients with sinusitis can be grouped according to the severity of their sinusitis into those with mild and those with severe anatomic evidence of sinusitis. The latter category includes those patients with significant anatomic anomalies, patients previously operated on who have substantial postoperative defects in the diseased areas, and those with significant paranasal sinus polyps. The remaining group with mild anatomic evidence of inflammation, which makes up the largest portion of those suffering from sinusitis, may nonetheless have significant and persistent symptoms despite undergoing medical therapies. Many patients are understandably resistant to traditional surgery, such as functional endoscopic sinus surgery (FESS), in particular if their symptoms are mild. Thus, that is the target group for non-invasive treatments. The goal is a procedure that is reliable, long lasting, pain free, safe, has no tissue removal, and allows an immediate return to full activities.

Development of non-invasive procedures requires an understanding of the anatomical features of the sinuses and the nasal cavity as well as an appreciation of the mucus drainage pathways.

The ostium of the maxillary sinus (MSO) is relatively invariant in the anterior inferior ethmoid infundibulum as outlined in my earlier submission. The boundaries of the ostium are also well-defined, as illustrated in FIG. 1. The anterior margin or wall of the MSO is comprised of relatively thick and rigid maxillary bone. The superolateral wall is the bony orbit itself. The inferomedial wall is the floor of the ethmoid infundibulum, comprised to varying degrees of thin uncinate bone, modestly thicker bone of the medial wall of the maxillary sinus or lateral wall of the nose—they are one and the same here or fibrous attachments between the other two bones, covered in each case with the overlying mucosa. The posterior margin abuts the posterior fontanelle of the maxillary sinus, so called because it is quite soft and flexible. This term is generally applied in anatomy to a membrane-covered opening within a bone or between bones, as in the “soft spots” of the young infant skull. Here, there is generally a gap in the bone so the “wall” posterior to the posterior margin generally consists of fibrous tissue and mucosa only, or very thin bone. It should be noted that this is the location of the greatest anatomic variability. If the MSO is large, it is usually this portion which is expansive, and posterior to the MSO but within the posterior fontanelle is also where one finds accessory ostia.

As illustrated in FIGS. 2(a) and 2(b), there are two basic configurations of the MSO. If it is small, such as less than 5 mm maximum diameter, it is ellipsoid, with the long axis oriented anteroposterior, FIG. 2(a). If it is larger than this, it will generally adopt a modified “figure eight” configuration with two ellipsoid spaces connected by a somewhat narrower isthmus. The “top” of the “eight” is anterior, and has the same shape and dimensions of the small type of MSO described above. The “bottom” of the “eight” is posterior and lies within the posterior fontanelle. As illustrated in FIG. 2(b), if one ellipsoid of the “eight” is larger than the other, it will generally be the posterior one.

The two described variants, with or without attendant accessory ostia within the posterior fontanelle, account for the vast majority of naturally occurring human MSOs. When the pattern varies, the variation is slight and does not alter the substance of any of the discussion below. Another key anatomic pattern of the MSO relates to its orientation. In most texts and discussions, the MSO is represented as roughly vertical. There are two distinct variants and they conveniently correspond precisely to those described in the preceding paragraph. The plane of the small ellipsoid type is more closely horizontal; that is, transverse. Generally the plane is slightly tilted with the lateral margin somewhat superior to the medial margin, as illustrated in FIG. 3.

These are hereafter referred to as the (“inferomedial” and “superolateral” margins). The configuration of the ellipse is such that, if there is a true long axis, the inferomedial and superolateral margins approach each other more closely than the anterior and posterior margins.

For those MSOs of the second anatomic configuration, the orientation is more complex. Here, the “eight” does not lie in one plane. The “top” of the “eight”, or the anterior ellipsoid, lies in the same plane as a small type MSO, or roughly transverse, with superolateral and inferomedial margins. At the isthmus, however, the plane orientation rotates and becomes vertical within the posterior fontanelle.

The physiology of the MSO and its mucociliary clearance have little variation as well. In naturally occurring MSOs, the pathway of mucus drainage from the sinus is quite narrow and the majority exits along the inferomedial margin of the MSO. Further, its anteroposterior location is well-defined. In small MSOs, the mucus exits the inferomedial margin of the ellipse, and then courses along the floor of the ethmoid infundibulum, and then angles along the lateral wall until its exit from the infundibulum posteriorly (FIG. 4(a)). Of further interest, in large MSOs of the “FIG. 8” type, most of the mucus still exits along the inferolateral margin. A high percentage of the mucus still exits from the anterior ellipse, illustrated in FIG. 4(b), as the cilia of the sinus largely beat in this direction. In essence, regardless of the MSO configuration, the bulk of the mucociliary clearance proceeds as if there is only an anterior small ellipsoid ostium, essentially independent of the posterior fontanelle component or any accessory ostia. This observation comports well with the clinical observation that the correlation between MSO size and the presence or severity of chronic maxillary sinusitis is slight or nonexistent.

In order to enlarge the pathways it is necessary to know where they are. Current treatment methods need improvement in that mucus exits the MSO through the ethmoid infundibulum and exits it posteriorly.

Current treatments do not take into account the microanatomy and physiology of flow. Further, inflammation plays a significant role in clinical chronic sinusitis. Nasal inflammation leads to swelling of the mucosa to varying degrees. If the diameter of a mucociliary pathway is sufficiently small the swelling should not be too large to impede flow. When the swelling of the mucosa causes the mucus of one wall to contact the mucus from the opposite wall mucociliary flow is drastically impeded.

With regard to the flow pathway from the MSO, the greatest risk is the superolateral and inferomedial walls become apposed. This is a result of the walls being close together, and the midpoint of the inferomedial margin is the exact spot of maximum mucociliary flow as illustrated in FIG. 5(a). This problem also exists for any MSO irregardless of size, when the superolateral and inferomedial margin approach each other closely in the anterior ellipsoid. Again this is regardless of any configuration of a posterior fontanelle component as shown in FIG. 5(b). As illustrated in FIG. 5(c) the risk is also present in any MSO where the adjacent ethmoid infundibulum is narrow. It is not the size of the MSO that is at issue, rather, it is the size of the micropathway of mucociliary clearance that must be addressed. Patients who suffer from mild forms of chronic maxillary sinusitis, and have failed appropriate medical therapies, are likely to benefit from surgeries that enlarge the MSO and/or enlarge the ethmoid infundibulum or remove the uncinate.

### SUMMARY OF THE INVENTION

An object of the present invention is to provide improved minimally invasive devices, systems and methods for accessing and treating maxillary sinusitis.

Another object of the present invention is to provide devices and methods for intranasal bone remodeling to treat maxillary sinusitis.

A further another object of the present invention is to provide minimally invasive devices, and methods for accessing and treating maxillary sinusitis without the superolateral and inferomedial walls becoming apposed.

Yet another object of the present invention is to provide minimally invasive devices, and methods for accessing and treating maxillary sinusitis.

Still another object of the present invention is to provide minimally invasive devices, and methods for accessing and treating maxillary sinusitis that addresses the size of the micropathway of mucociliary clearance.

Another object of the present invention is to provide minimally invasive devices, and methods for accessing and treating maxillary sinusitis for any MSO regardless of size.

Yet another object of the present invention is to provide minimally invasive devices, and methods for accessing and treating maxillary sinusitis regardless of any configuration of a posterior fontanelle component.

Yet another object of the present invention is to provide minimally invasive devices, and methods for accessing and treating maxillary sinusitis in any MSO where the adjacent ethmoid infundibulum is narrow.

These and other objects of the present invention are achieved in, a method for accessing and treating at least one of a maxillary sinusitis, sphenoid sinusitis and frontal sinusitis. A perforation is created in an uncinate via an anterior keyhole. A position of an MSO is verified using a probe. The probe is used to verify dimensions of the MSO. Verification is made that an anteroposterior length of the ostium is sufficient. A targeted medial displacement of a medial wall of the maxillary sinus and uncinate is made using an uncinate medializing device.

In another embodiment of the present invention, an apparatus accesses and treats maxillary sinusitis, sphenoid sinusitis and frontal sinusitis. First and second distal flanges are coupled with or include first and second opposing jaws. A shaft is coupled to the first and second distal flanges. One or more handles are coupled to the shaft.

### BRIEF DESCRIPTION OF THE DRAWINGS

FIG. 1 illustrates the boundaries of the ostium.

FIG. 2(a) illustrates the frontal sinus itself and the frontal contribution to the skull base; and inferiorly, the nasal cavity just anterior to the ethmoid bulla.

FIG. 2(b) illustrates directed anterolateral displacement of the posteromedial lamina of the superiormost cell of the agger nasi system in one embodiment of the present invention.

FIG. 3 illustrates that the plane of the small ellipsoid type is more closely horizontal; that is, transverse, and generally the plane is slightly tilted with the lateral margin somewhat superior to the medial margin.

FIG. 4(a) illustrates that the mucus exits the inferomedial margin of the ellipse, courses along the floor of the ethmoid infundibulum, and then angles along the lateral wall until its exit from the infundibulum posteriorly.

FIG. 4(b) illustrates that a high percentage of the mucus still exits from the anterior ellipse as the cilia of the sinus largely beat in this direction.

FIG. 5(a) illustrates that the midpoint of the inferomedial margin is the spot of maximum mucociliary flow as illustrated in FIG. 5(a).

FIG. 5(b) illustrates the risk of the superolateral and inferomedial walls become apposed for any MSO, irregardless of size, when the superolateral and inferomedial margin approach each other closely in the anterior ellipsoid.

FIG. 5(c) illustrates that the risk is also present in any MSO where the adjacent ethmoid infundibulum is narrow.

FIG. 6(a) illustrates one embodiment of the device of the present invention, with one jaw convex in cross-section, and the other jaw with a complementary concavity.

FIG. 6(b) illustrates that the surgeon provides manual force to the device of FIG. 6(a), bringing the jaws together and transfixing the intervening sinus lamina between them.

FIG. 6(c) illustrates that upon release by the FIG. 6(a) device the lamina retains the shape induced by the interlocking fit of the two jaws.

FIGS. 6(d) through 6(f) illustrate another embodiment of a device of the present invention.

FIGS. 6(g) through 6(i) illustrate adjacent opposing forces can be created.

FIGS. 7(a) and 7(b) illustrate a perforation created in the uncinate.

FIGS. 8(a) and 8(b) illustrate in one embodiment that a created perforation is about 1-3 mm.

FIGS. 9(a) and 9(b) illustrate verification of the approximate dimensions of the MSO.

FIG. 10(a)-(f) in one embodiment of the present invention, once the position and size of the MSO are verified the surgeon proceeds with targeted medial displacement of the medial wall of the maxillary sinus and uncinate using an uncinate medializing device.

FIGS. 11(a) and 11(b), in one embodiment a distal assembly includes a distal mobile flange that is roughly elliptical in cross section.

FIGS. 12(a) through 13(b) illustrate a remodeling of the uncinate in a controlled fashion to a more medial for mucociliary flow position.

FIGS. 14(a) and 14(b) illustrate an embodiment of the present invention where a forward-opening device can be introduced anterograde via the keyhole instead of via the hiatus semilunaris.

FIGS. 15(a) through 15(c) illustrate an embodiment of a device of the present invention with an angled distal assembly.

FIGS. 16(a) through 16(b) illustrate an embodiment of the present invention where the distal flange s introduced into the MSO via the keyhole and its free end is directed inferiorly.

FIGS. 17(a) and 17(b) illustrate an embodiment of the present invention whereas the mobile and fixed flanges engage; the medial wall of the maxillary sinus/lateral wall of the nose is transfixed between them and is compressed into the curviplanar shape of the device surfaces.

FIGS. 18(a) and 18(b) illustrate an embodiment of the present invention where the inferomedial rim of the MSO is displaced from its superomedial rim.

FIGS. 19(a) through 19(d) illustrate an embodiment of the present invention where the sphenoid ostium (SSO) is found, with negligible anatomic variation, on the anterolateral surface of the sphenoid rostrum.

FIGS. 20(a) through 20(b) illustrate an embodiment with directed anterolateral displacement of the posteromedial lamina of the superiormost cell of the agger nasi system, displacing the anterolateral wall of the frontal recess further anterolateral, opening it without manipulating the more hazardous posteromedial wall of the recess.

FIGS. 20(c) and (d) illustrate an embodiment of the present invention where the recess can be opened without manipulating the posteromedial wall.

FIGS. 21(a) through 21(d) illustrate an embodiment of the present invention where drainage from most of the anterior ethmoid can be relieved by opening the EBO and HSS, accomplished simultaneously by creating anterior displacement of the medial margin of the EBO using adjacent opposing forces to the lamina of the ethmoid bulla anteromedial to the EBO.

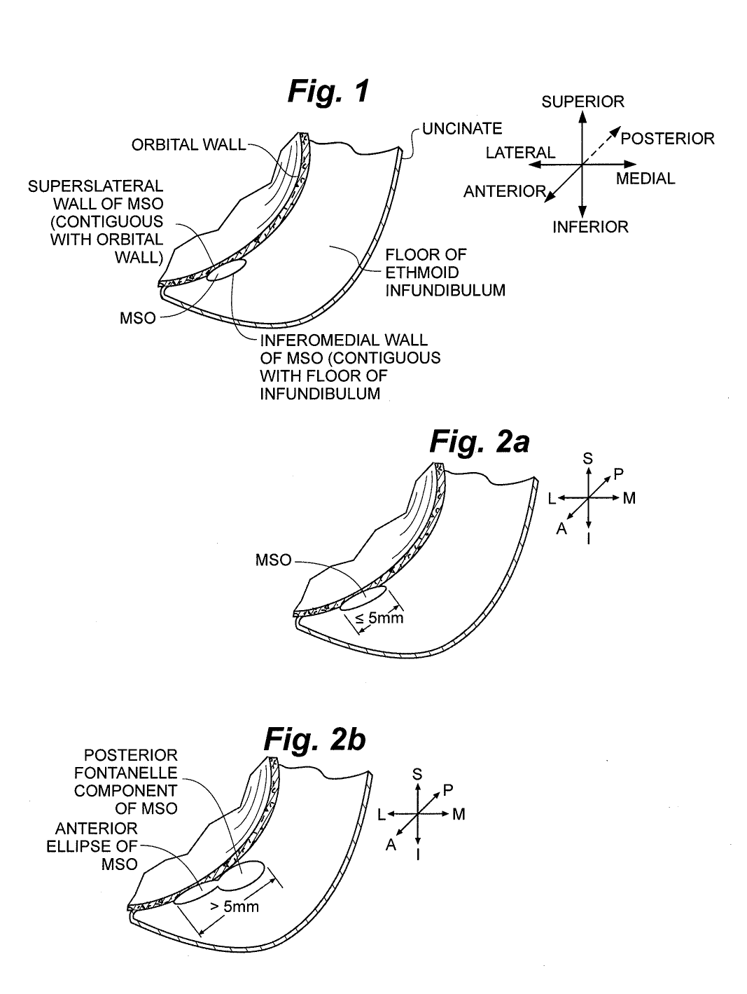

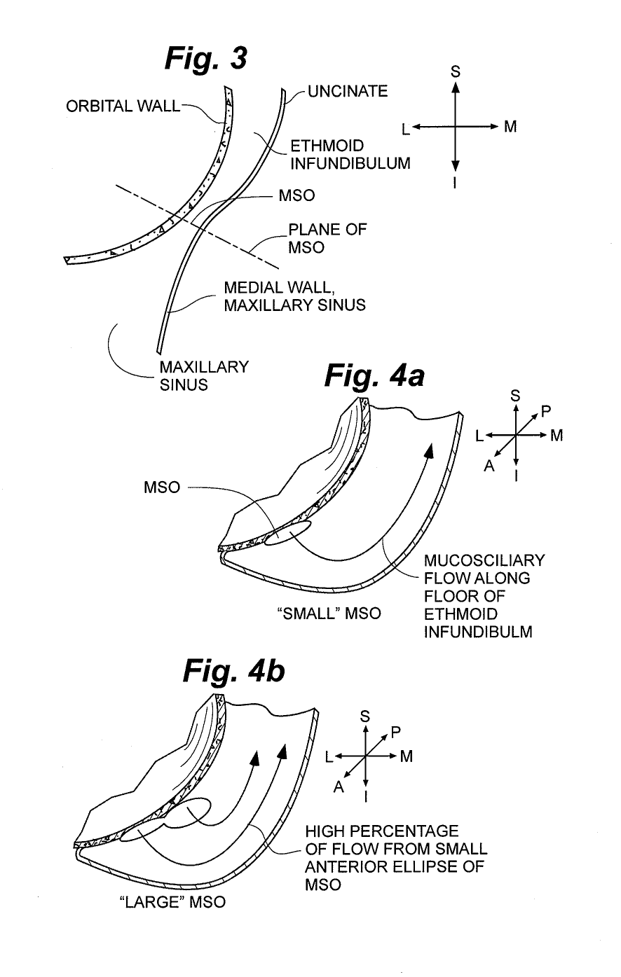

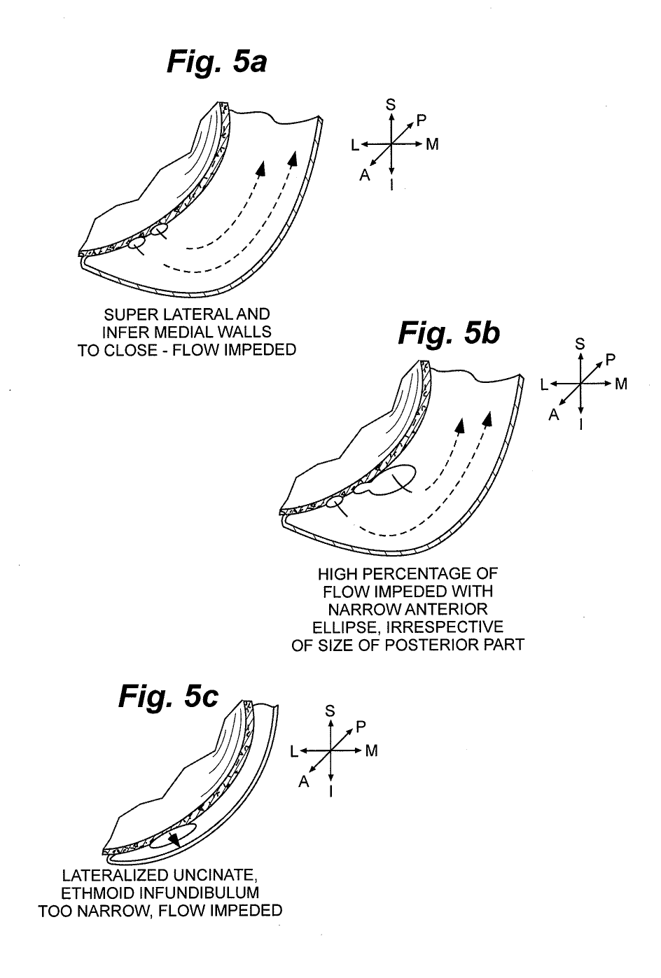

### DETAILED DESCRIPTION OF THE PREFERRED EMBODIMENTS

In various embodiments, the present invention provides minimally invasive devices, systems and methods for accessing and treating maxillary sinusitis.

Because the micropathway is essentially anatomically invariable and predictable from patient-to-patient, the present invention, (i) reliably addresses the true MSO and not an accessory ostium, (ii) reliably separates the superolateral and inferomedial margins within the boundaries of the anterior ellipsoid, and (iii) reliably separates the lateral and medial walls of the ethmoid infundibulum. The devices and methods of the present invention achieves this without irreversible trauma to the mucosa of the micropathway, and preferably with at most transient small scale bleeding and of 50 ml or less and minimal or no post-operative pain. The device and methods of the present invention achieve this without manipulating the orbital wall, anterior margin of the MSO, or, necessarily, the posterior margin and, specifically, without radial dilation of the MSO and ethmoid infundibulum. In one embodiment, there is about a 3 mm of increase in diameter without manipulation of the indicated areas.

In one embodiment, a method is provided that includes the keyhole 16 approach described Ser. No. 12/804,398, fully incorporated herein by reference.

In one embodiment of the present invention, targeted bone remodeling is done. For purposes of the present invention, the bone remodeling is to generate fracturing or dislocation of a discretely chosen mucosally lined bone lamina within the nose and paranasal sinuses, causing a permanent displacement or reshaping of the lamina in the interest of augmenting mucociliary clearance through an adjacent pathway. More particularly, mucosa-covered bone walls are chosen for their proximity to the micropathway and manually using surgical devices manipulated by the performing practitioner and distorted using devices of the present invention. In one embodiment of the method, the medial wall of the maxillary sinus is targeted first so as to specifically displace the inferomedial margin of the MSO further inferomedially. This targeted displacement is accomplished using a device of the present invention. Subsequently, in one embodiment of the present invention, the inferior limb of the uncinate process is displaced medially. In one embodiment, this displacement is about 1.5-3 mm and in one embodiment about 2-5 mm. It will be appreciated that the displacement number can be dependent on the specifics of patient anatomy: deformability, unique attachment points of the manipulated structures, and the like. In some patients, it may be desirable to displace the posterior margin of the MSO further posterior via the keyhole 16 about 1.5-3 mm and in one embodiment about 2-5 mm. It is not desirable to manipulate the anterior margin of the MSO, superolateral margin of the MSO or orbital wall, nor the more superior segments of the uncinate. A balloon catheter device has the undesirable effect of creating these types of manipulation because they push outward radially in all directions, and to a fixed diameter, typically 5-7 mm, irrespective of the anatomy of the individual. With the methods of devices of the present invention, permanent deformation is achieved at only those sites chosen by the surgeon, and only to the degree desired.

Second, with current balloon devices, one cannot easily customize the degree of deformation from lamina-to-lamina or sinus-to-sinus within the same patient, as balloon dimensions are fixed and it would be cumbersome and cost-prohibitive [and therefore, negatively incentivized] to use an array of different sized balloons to optimize the procedure for a given patient. The devices and methods of the present invention allow the surgeon to suit the amount of displacement to the unique characteristics of each patient and each lamina within the patient by enabling him to exert whatever force is needed at precisely whatever site is chosen to achieve the precise displacement desired.

Third, the balloon devices cause collateral deforming pressure to adjacent laminae of the skull which is undesirable. More particularly, the pressure is applied to the orbit in maxillary sinus balloon procedures and to the skull base, separating the brain from the nose in frontal sinus balloon procedures. The methods and devices of the present invention avoid such manipulations by allowing the surgeon to apply force to those laminae, and only those laminae, whose displacement or deformation is desirable to restore mucociliary clearance. This is achieved without any manipulation of the adjacent structures.

With the methods and devices of the present invention, specific knowledge of the mucociliary micropathway is used. This knowledge enables the practitioner to target specific laminae of each ostium with a directed compression strategy. The methods and devices of the present invention provide derivative strategies for the micropathway of the maxillary and other sinuses and use adjacent opposing forces to either face of the lamina in question to generate a new convexity by fracturing the thin intervening bone.

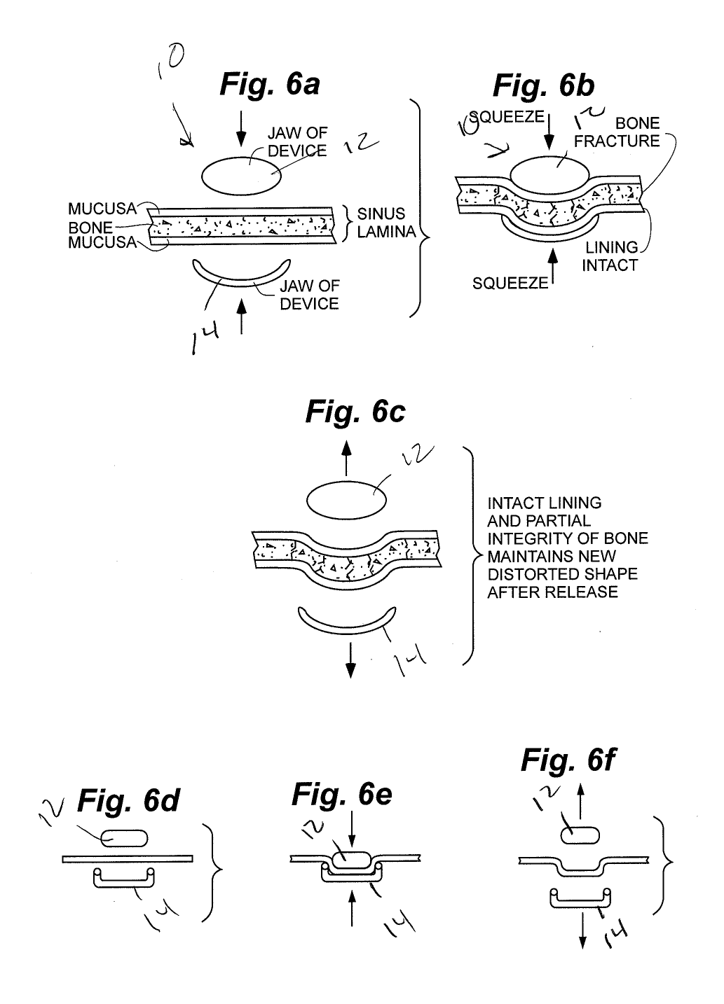

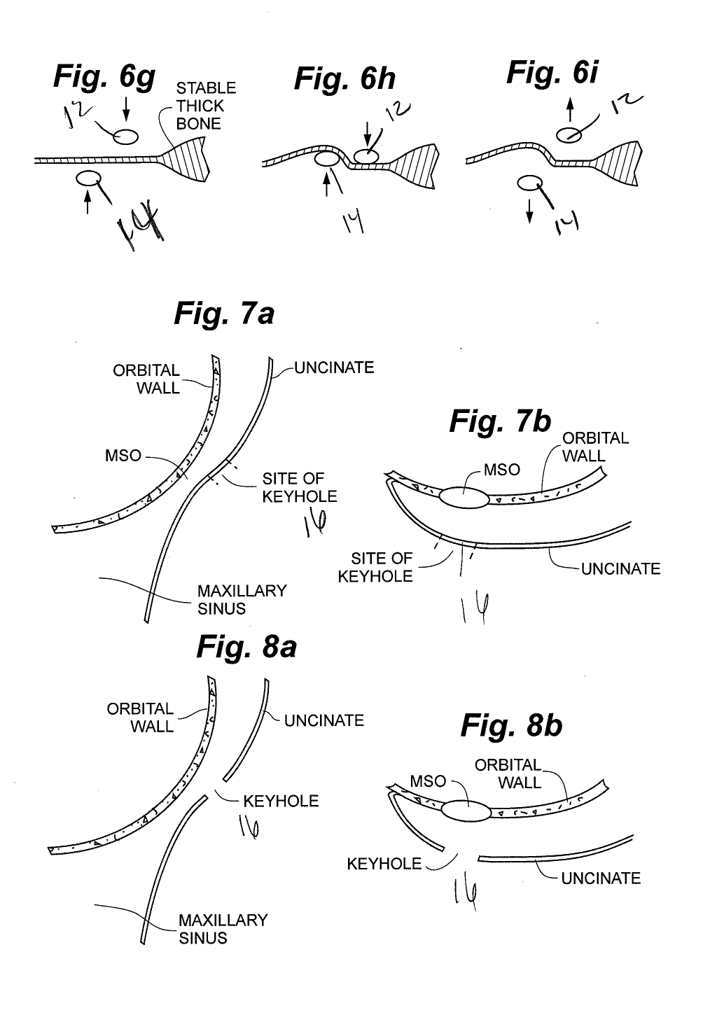

In one embodiment, illustrated in FIG. 6(a), an embodiment of the device 10 of the device of the present invention has two jaws 12 and 14, are on either side of a typical sinus lamina. In one embodiment, one such jaw 12 is convex in cross-section; the other jaw 14 has a complementary concavity. The surgeon provides manual force to the device 10 bringing the jaws 12 and 14, together and transfixing the intervening sinus lamina between them (6b). The jaws 12 and 14 are constructed of a suitably rigid, noncompressible material such as surgical grade steel and therefore induce multiple microfractures to the intervening bone of the sinus lamina. Upon release by the device, said lamina retains the shape induced by the interlocking fit of the two jaws (6c).

FIG. 6(d) through 6(f) illustrate another embodiment of a device 10 of the present invention, in which there is no “puzzle piece” fit between the jaws 12 and 14. A similar effect is obtained by modifying the second jaw 14 to have specific pressure points on the opposite side of the intervening lamina on either side of and adjacent to the pressure point exerted by the first jaw 12 FIG. 6(d). When the two jaws 12 and 14 are brought into compression and transfix the intervening sinus lamina, FIG. 6e), the effect, FIG. 6(f) is quite similar to that in FIG. 6(a) since in both embodiments, the relevant forces are directed to opposite surfaces of the lamina but, instead of direct opposition, they are immediately adjacent. Thus, deformation is induced to the planar structure within. Adjacent opposing forces can also be created, as in shown in FIGS. 6(g) through 6(i), by the use of fixed rigid bone as one of the opposing forces. As depicted in FIG. 6(g), the two jaws 12 and 14 are similarly ellipsoidal in cross-section. The intervening targeted lamina is fixed in space at one edge by its attachment to rigid bone that is not deformable within the stress force range of the device. Thus, nearby forces are not sufficient to move this attachment. As the jaws 12 and 14 are squeezed into opposition in scissors-like fashion by the practitioner, the fixed attachment acts as an adjacent opposing force, assisting in deformation at the site of jaw opposition FIG. 6(h) and FIG. 6(i). In this embodiment, the fixed bone attachment creates the adjacent opposing force generated of FIGS. 6(a) through 6(f) by one of the pressure points of the second jaw 14. The force is analogous to the force generated by a wall when one pushes against it. If the wall doesn't move, it must exert an equal force to the push.

It will be appreciated that the devices and methods of the present invention for the maxillary sinus micropathway have analogues for the sphenoid, frontal, and specific locations of the anterior ethmoid.

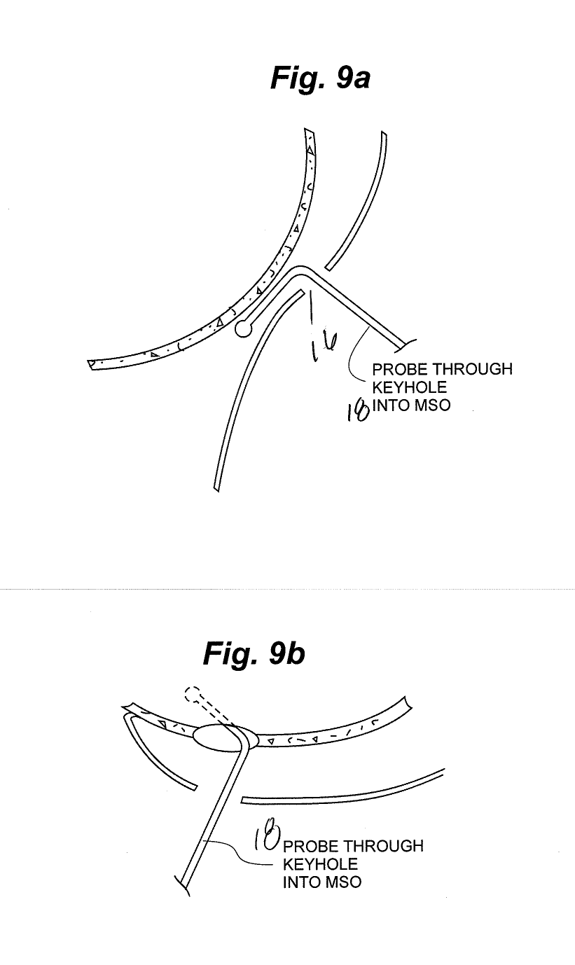

In one embodiment of the present invention, a perforation is created in the uncinate according to the anterior keyhole 16 approach of Ser. No. 12/804,398, fully incorporated herein by reference, and as illustrated in FIGS. 7(a) and (b). In one embodiment, the perforation is about 0.5-3.5 mm, in another embodiment, it is about 1-3 mm, as illustrated in FIGS. 8(a) and (b). The position of the MSO is verified with a blunt probe 18 such as a sinus seeker. Using this probe 18, one can verify the approximate dimensions of the MSO, as illustrated in FIG. 9. The physician then can verify that the anteroposterior length of the ostium is adequate, roughly 5 mm or greater, and this will usually be the case.

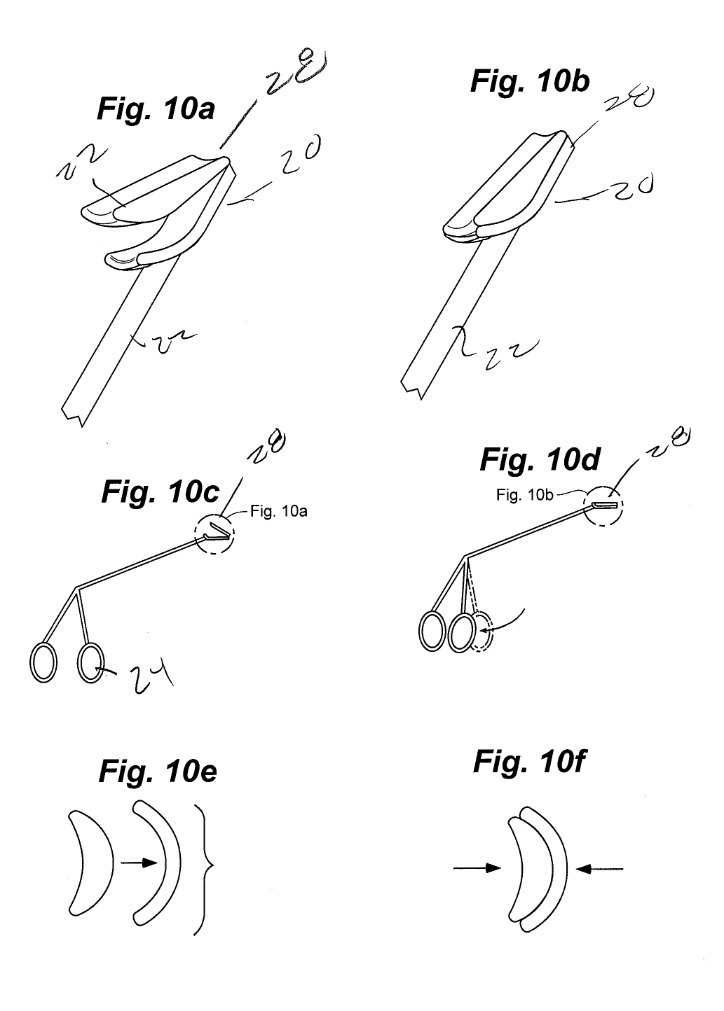

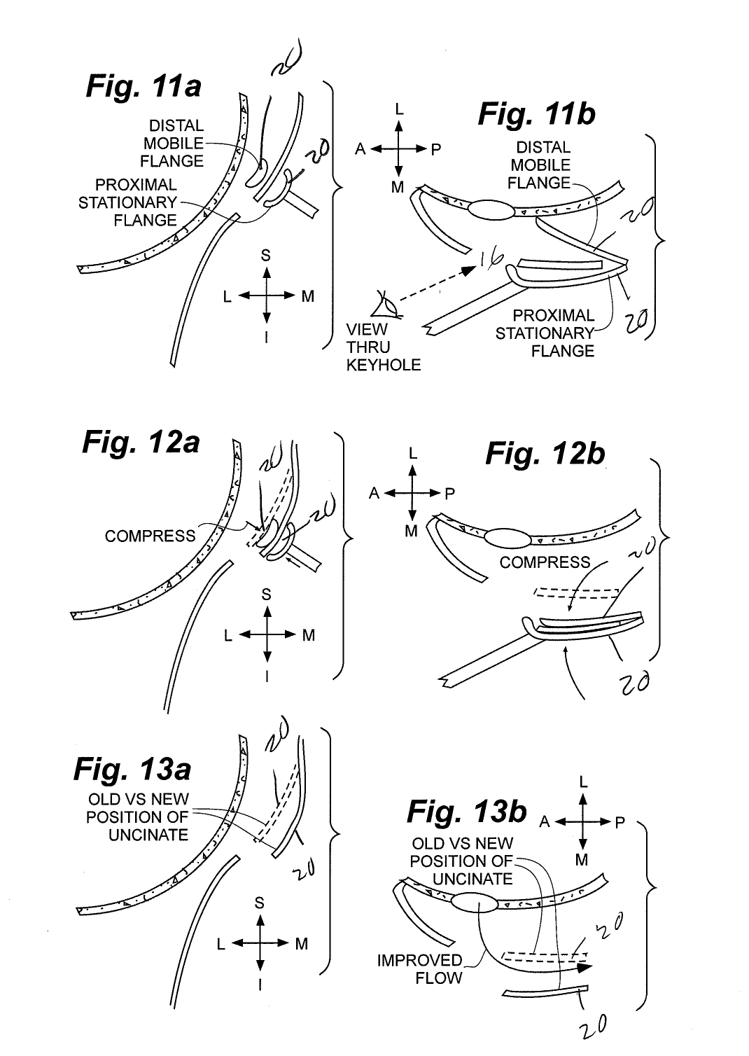

Once the position and size of the MSO are verified, the surgeon proceeds with targeted medial displacement of the medial wall of the maxillary sinus and uncinate using an uncinate medializing device as illustrated in FIG. 10. The device 20 of FIGS. 10(a)-(d) largely resembles the backbiting hole punch from Ser. No. 12/804,398 and includes a distal assembly of flanges 20 that manipulate the tissue mounted on a shaft 22 that traverses the nostril and transmits the action of the handles 24 manipulated by the surgeon's dominant hand, FIGS. 10(a)-(d). Squeezing the handles 24 together brings the flanges 22 of the distal assembly together transmits the force of the squeeze to within the stress limits of the material, FIGS. 10 [symbol] and 10(f). In one embodiment, the device 20 is made of a rigid material suited to surgical instruments, including but not limited to, stainless steel and the like. In one embodiment, a distal assembly 28 includes a distal mobile flange 20 that is roughly elliptical in cross section and is, ideally, convex in surface with respect to the proximal fixed flanges, FIGS. 10(a), 10(b) and 11(a) and 11(b). As non-limiting examples, the length of the flanges is in the range of 5-20 mm, 7-15 mm, and can be about 11.5-12.5 mm, with an ideal near 12 mm. The typical width of each flange can be about 0.5-5.5 mm and, like the length, can require a few different ideal dimensions, in order to suit itself to unique patient anatomy.

In one embodiment of the device, a single proximal flange is concave in surface with respect to the opposing distal flange, FIGS. 10(a), 10(b), 11(a) and 11(b). The radii of the axes of the ellipsoid cross section of the distal flange 20 can be about 1.5-4.5 mm, about 2-4 mm, and the like, and the length of the distal flange 20 can be 2.5-20.5 mm, 3-20 mm 0.5-1.5 cc and about 1 cm. These measurements are replicated for the proximal flange 20 so that the two flanges mesh seamlessly when brought into apposition by squeezing the handles 26 together. In one embodiment, a small gap 0.5-1.5 mm and about approximately 1 mm is maintained between the flanges in order to accommodate the thickness of the uncinate in compression, but without actual tissue penetration. In one embodiment, a long axis of the flanges 20 is directed along the axis of the shaft 24 though an angulation of roughly 0.4-40.5 degrees, 15-25 degrees, 19-21 degrees and 20 degrees with respect to the shaft. In one embodiment, it is desirable to have a better accommodation with the intranasal anatomy, as the axis of approach from nostril to the surgical site is about 20 degrees displaced from the axis of the ethmoid infundibulum. As in the backbiting hole punch, the flanges 20 open toward the handle. This embodiment allows for insertion of the instrument's mobile flange into the open posterior space of the ethmoid infundibulum, as illustrated in FIG. 11.

Similar to the hole punch disclosed in Ser. No. 12/804,398, the tip of the mobile flange can be advanced anteriorly up to the anterior attachment of the ethmoid, with the fixed flange immediately medial to it on the opposite side of the uncinate as illustrated in FIGS. 11(a) and 11(b). The position of the tip of the mobile flange 20 may be visually assessed through the keyhole 16, if one has been created, though this is not necessary. The jaws 12 and 14 are brought together, transfixing but not cutting or tearing the uncinate or its attachment, and by fracturing the underlying paper-thin bone and attachment suture. This remolds the uncinate in a controlled fashion to a more medial for mucociliary flow position, as illustrated in FIGS. 12 and 13. In various embodiments, displacement of the targeted lamina site can be 1.5-8.5 mm, 2-8 mm, 2.5-4.5 mm and about 3-4 mm.

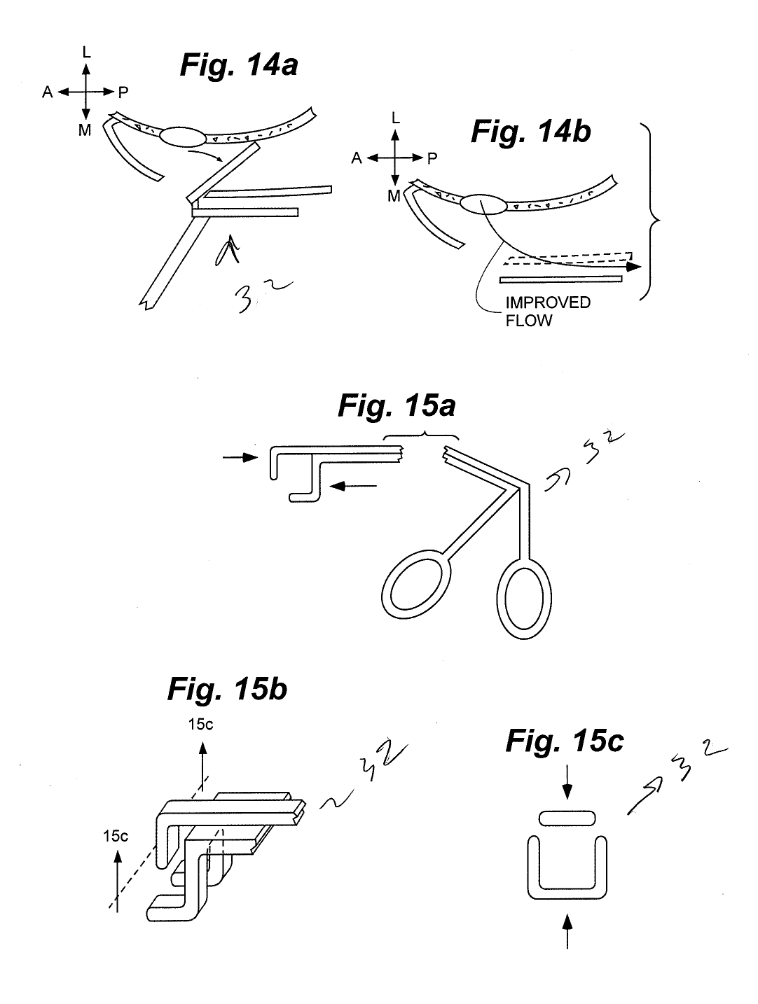

An analogous forward-opening device 30 can be introduced anterograde via the keyhole 16, as illustrated in FIGS. 14(a) and 14(b), rather than as outlined above with the backbiting device retrograde via the hiatus semilunaris, if desired the same effect can be attained. Either device 10 or 30 can be used with the methods of the present invention as an adjunct to improve access to balloon dilate the MSO if that is desired. It can be cumbersome, traumatic, or impossible, in some cases, to introduce balloon catheter devices through the posterior exit of the ethmoid infundibulum without expanding it. The methods and devices of the present invention accomplish this objective. In any event, the actual configuration of the MSO and the space between the uncinate/inferomedial rim of the MSO and superolateral (orbital) wall can now be assessed with routine blunt-tipped probes of the known sinus art and/or visually. If the space is adequate, typically 3 to 8 mm, though generally 3 to 5 mm would be expected, the procedure is complete. If deemed inadequate, the space can be enlarged with the medial maxillary crimping device 32.

The device 32 illustrated in FIG. 15(a) resembles the above device in configuration except that the flanges 20 of the distal assembly are angled at about 45 to 90 degrees, 80 to 90 degrees, substantially perpendicularly with respect to the shaft, in the general direction of the handles 26. This angulation directs the flanges 20 inferiorly when directed into the nose. In one embodiment of the device 32, there is a single distal flange 20. In one embodiment, it is convex in surface with respect to the opposing proximal flange 20. The radii of the axes of the ellipsoid cross section of the distal flange are approximately 2-4 mm and the length of the distal flange 20 is from 3-20 mm. In one specific embodiment, it is less than 1 cm. These measurements are replicated for the proximal flange 20 so that the two flanges 20 mesh when brought into apposition by squeezing the handles 26 together. In another embodiment of the device 32 illustrated in FIG. 15(b) two proximal fixed flanges 20 are separated by a distance just large enough, that can be about 3-8 mm, to admit the distal flange 20 between them, when brought into apposition by squeezing the handles 26 together. Other similar configurations are possible and may be desirable, such as analogous configurations set forth with respect to the FIGS. 6(a), (b) and (c) embodiments. The dimensions are otherwise similar to those of the distal flange 20. In some embodiments, it may be desirable to coat the distal flange 20 and possibly the proximal flange(s) 20 with a softer material so as to minimize pressure to the target tissues during use. A variety of different coatings can be employed, including but not limited to synthetic rubber, plastic, and the like. Such coatings are used in lid retractors for ophthalmic surgery, for example. In some embodiments, it may also be desirable to angle the shaft 24 by approximately 20-45 degrees to the left or right near the distal assembly 20 to accommodate the intranasal anatomy.

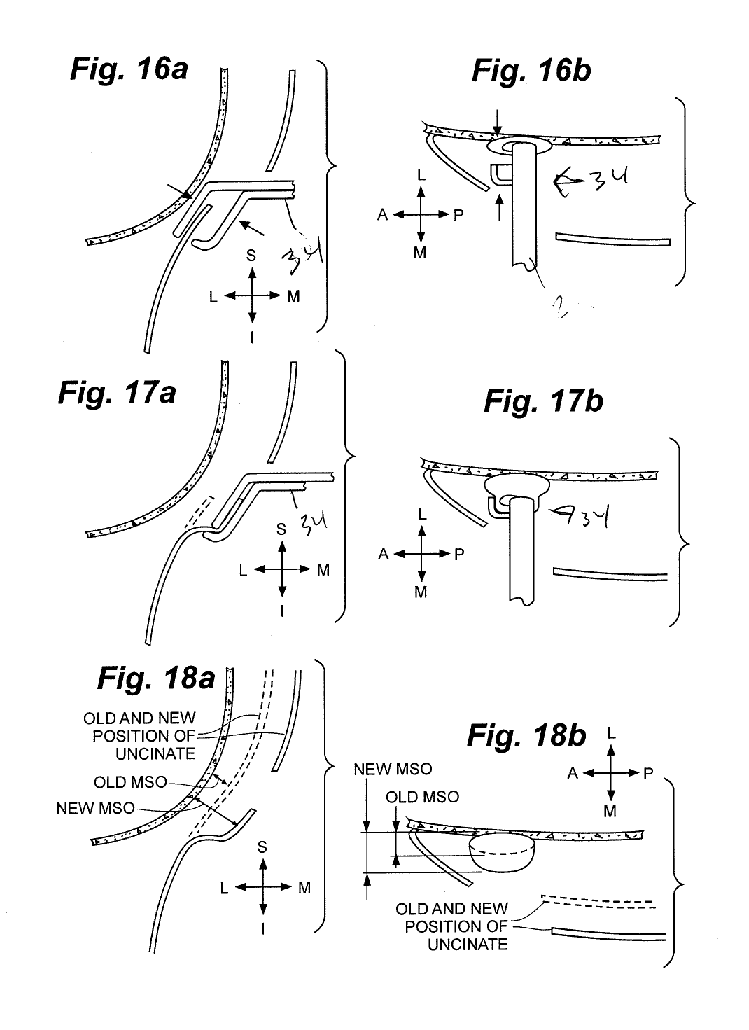

In one embodiment of the method of the present invention, the distal flange 20 is introduced at this time into the MSO via the keyhole 16 and its free end is directed inferiorly, as illustrated in FIGS. 16(a) and 16(b). The instrument 34 is then advanced a few more millimeters, as a non-limiting example, 3 to 12 mm, into the nose such that the fixed proximal flange(s) 20 engage the lateral wall of the nose. The flanges are then drawn anteriorly as far as they will go. This action seats the distal assembly 20 in the anterior ellipse of the MSO. The handles 26 are then squeezed together gradually but forcibly. As the mobile and fixed flanges 20 engage, the medial wall of the maxillary sinus/lateral wall of the nose is transfixed between them and is compressed into the curviplanar shape of the device surfaces illustrated in FIGS. 17(a) and 17(b), displacing the intervening inferomedial margin/wall of the MSO medially. In various embodiments, the displacement can be about 1-10 mm, 1.5-9 mm, 2-8 mm, and the like. This maneuver further displaces the inferomedial rim of the MSO from its superomedial rim as illustrated in FIGS. 18(a) and 18(b). It various embodiments the displacement can be about 1-9 mm 2-8 mm or so, and the like. The effect is easily accessed via the keyhole 16. It should be noted that the keyhole 16, or other transuncinate access to the MSO, can be mandatory for performing the maneuvers during this portion of the method. The devices and methods of the present invention can be used in the anterior ethmoid (via the hiatus semilunaris superior), in the frontal (via its recess), and in the sphenoid (via its ostium).

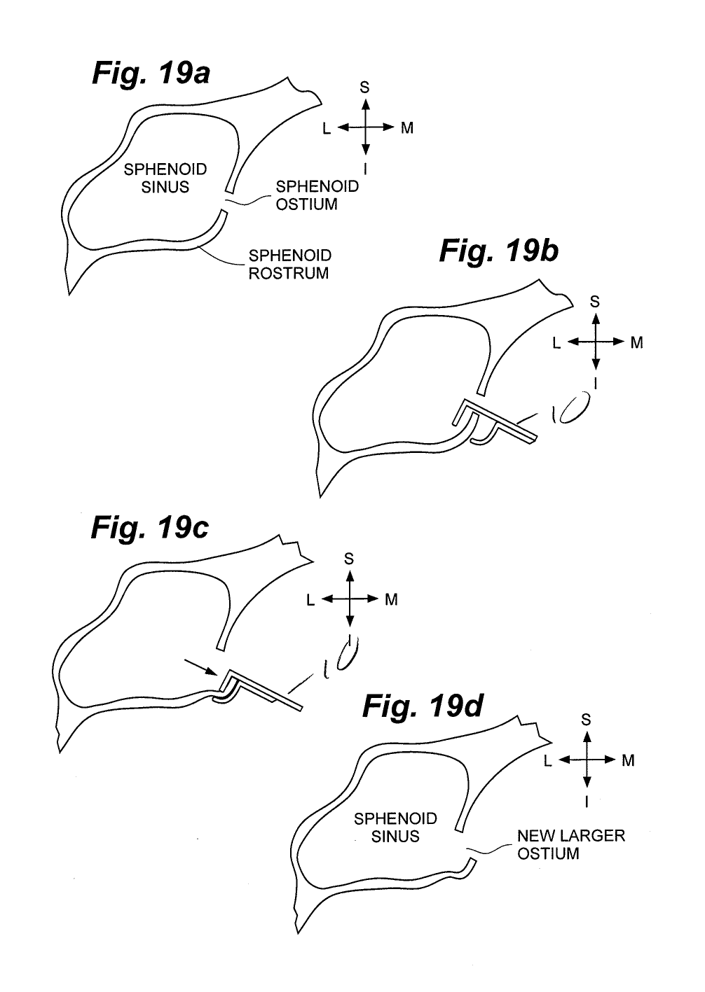

The sphenoid ostium (SSO) is found, with negligible anatomic variation, on the anterolateral surface of the sphenoid rostrum, the anterior most projection of the sphenoid bone into the nose, FIGS. 19(a)-19(d). It is generally found just posterior, superior, and medial to the body of the superior turbinate. In the intact sinonasal system, approach to the sphenoid ostium is via the main nasal cavity, remaining medial to the middle and superior turbinates until the sphenoid rostrum is reached. If the ostium is not immediately visible, the body of the superior turbinate can be gently reflected laterally and it can then be seen directly in most cases. The ostium can also be approached transethmoidally—that is, lateral to the middle turbinate—if one performs routine endoscopic resection of the bulk of the anterior and posterior ethmoid cells. Again, the ostium is then identified by gentle lateral reflection of the body of the superior turbinate. Unlike that of the maxillary described above, the sphenoid ostium opens directly into the nose rather than a secondary space, FIGS. 19(a)-19(d).

Once the sphenoid ostium is encountered, it may be enlarged in directed fashion using a device of the present invention. The mobile portion of the sphenoid device is identical to that of the maxillary device noted above and depicted in FIGS. 15(a)-(c). The sphenoid device is distinguished from the maxillary in that its shaft is either straight or angled up to 30 degrees in the vertical dimension, to accommodate the approach to the sphenoid rostrum.

Analogous to the maxillary ostium procedure, in one embodiment of the method of the present invention, at this time the distal flange 20 is introduced into the SSO under direct vision and its free end is directed inferiorly as illustrated in FIG. 19(b). The instrument is then advanced a few more millimeters, e.g., 3-12 mm, into the nose such that the fixed proximal flange(s) 20 engage the surface of the sphenoid rostrum. The handle 26 is then squeezed together gradually but forcibly. As the mobile and fixed flanges 20 engage, the anterior wall of the sphenoid sinus is transfixed between them and is compressed into the curviplanar shape of the device surfaces illustrated in FIG. 19(c) displacing the intervening inferior margin/wall of the SSO anteriorly about 2-8 mm or so. This maneuver further displaces the inferior rim of the SSO from its superior rim as illustrated in 19(d) which can be about 2-8 mm or so, enlarging the functional pathway from the SSO.

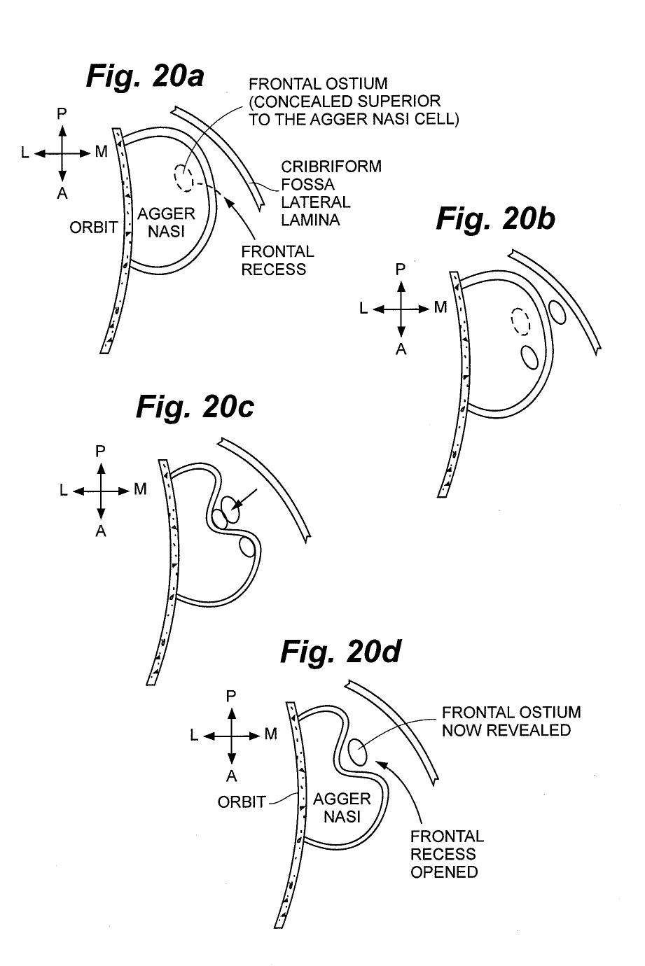

Both the frontal sinus and anterior ethmoid have predictable drainage pathways as well. The frontal sinus drains via the frontal recess. The space is found superior to the ethmoid bulla and posterior to the superiormost cell of the agger nasi system of the anterior ethmoid, in which there is some variation that will not be discussed in detail here. The boundaries of the space, therefore, are: anterolaterally, the posteromedial wall of the superiormost cell of the agger nasi; posteromedially, the frontal and ethmoid bone contributions to the skull base, a thin bony lamina separating the frontal recess from the intracranial cavity; superiorly, the frontal sinus itself and the frontal contribution to the skull base; and inferiorly, the nasal cavity just anterior to the ethmoid bulla (FIG. 20(a).

The methods and devices of the present invention avoid manipulation of the thin bone of the skull base, as perforation of this boundary leads to a significant complication, cerebrospinal fluid rhinorrhea. The devices and methods of the present invention can avoid this by directed anterolateral displacement of the posteromedial lamina of the superiormost cell of the agger nasi system, FIG. 2(b)-(d). This maneuver displaces the anterolateral wall of the frontal recess further anterolateral, opening it without manipulating the more hazardous posteromedial wall of the recess. In this method of the present invention, the lateral attachment of the anterolateral wall of the frontal recess is used as an adjacent opposing force to the application of anterolateral pressure with instrumentation to the anterolateral wall of the recess (FIG. 20(b)-(c), permanently remodeling it into a more anterolateral position, FIG. 20(c) (d), opening the recess without manipulating its posteromedial wall, the skull base.

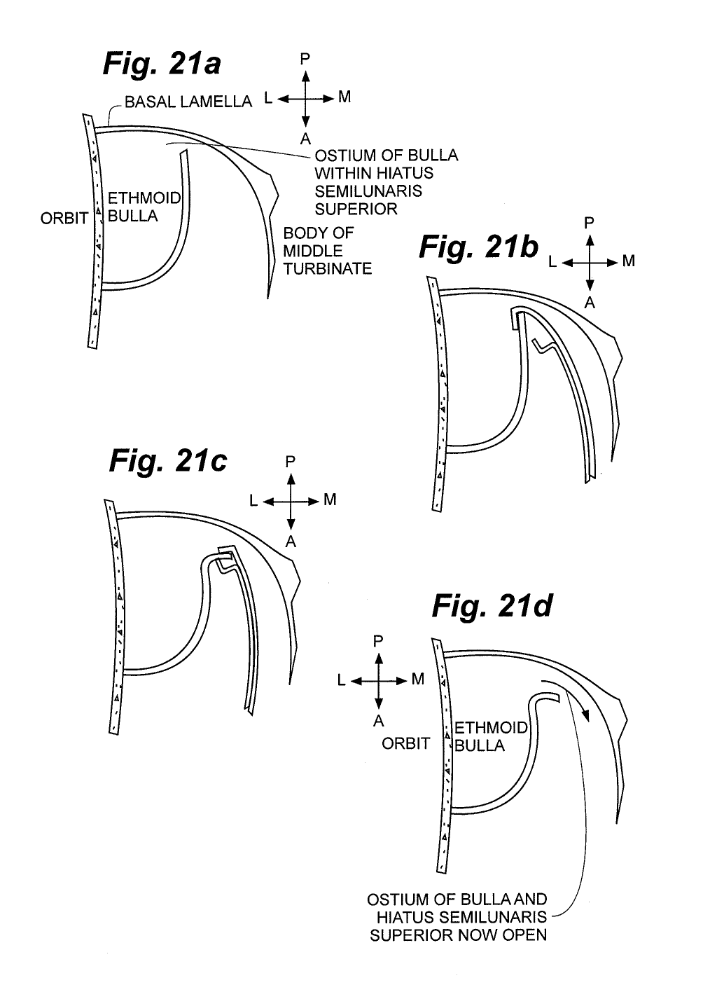

The cells of the anterior ethmoid found outside of the infundibulum and agger system drain into the ethmoid bulla, which then drains into the nose via the ethmoid bulla ostium (EBO) within the hiatus semilunaris superior (HSS), as described in detail in my previous submission. Drainage from most of the anterior ethmoid can thus be relieved by opening the EBO and HSS. Both can be accomplished simultaneously by creating anterior displacement of the medial margin of the EBO using analogous methods of adjacent opposing forces to the lamina of the ethmoid bulla anteromedial to the EBO as shown in FIG. 21(a)-(d).

The devices and methods of the present invention all use the principle of adjacent opposing forces applied to a select lamina of the sinonasal system. Each lamina described above is chosen because its permanent displacement remodels a specific known drainage pathway of the sinus system. In all cases, the method distinguishes from prior art. In the case of balloon catheters, the methods of the present invention distinguish by: limiting its manipulation to a selected lamina, without manipulating nearby collateral laminae that gain no benefit; avoiding an arbitrarily chosen fixed dilation diameter; preserving control of the degree of displacement of the chosen lamina; offering a means of dilating the drainage pathway of the ethmoid bulla. The method of the present invention distinguishes from other prior sinus art in that it is nonresective; that is, there is in general no need for any removal of soft tissue or bone to achieve its drainage objectives or, as in the keyhole 16 approach, any tissue removal is entirely negligible.

It will be appreciated that the methods and devices of the present invention can be used in tandem with any known and commercially available sinus methods and instruments. As noted earlier, methods and devices of the present invention, designed to mobilize the uncinate process, can be useful to further enable use of balloon catheters for maxillary sinus ostium dilation. Similarly, one might choose to resect portions of the ethmoid before employing methods of the present invention to open the frontal recess or sphenoid ostium. In these scenarios, the methods and devices of the present invention might be utilized to complement and augment the effectiveness of methods and devices of the prior art as well.

The foregoing description of various embodiments of the claimed subject matter has been provided for the purposes of illustration and description. It is not intended to be exhaustive or to limit the claimed subject matter to the precise forms disclosed. Many modifications and variations will be apparent to the practitioner skilled in the art. Particularly, while the concept “component” is used in the embodiments of the systems and methods described above, it will be evident that such concept can be interchangeably used with equivalent concepts such as, class, method, type, interface, module, object model, and other suitable concepts. Embodiments were chosen and described in order to best describe the principles of the invention and its practical application, thereby enabling others skilled in the relevant art to understand the claimed subject matter, the various embodiments and with various modifications that are suited to the particular use contemplated.

What is claimed is:

1. A method is provided for accessing and treating at least one of a maxillary sinusitis, sphenoid sinusitis and frontal sinusitis, comprising:
creating a perforation in an uncinate with via an anterior keyhole;
verifying a position of an MSO using a probe;
using the probe to verify dimensions of the MSO; and
performing a targeted medial displacement of a medial wall of the maxillary sinus and uncinate using an uncinate medializing device.

2. The method of claim 1, further comprising:
verifying that a anteroposterior length of the ostium is sufficient;

3. The method of claim 1, wherein the perforation is 0.5-3.5 mm.

4. The method of claim 1, wherein the probe is a sinus seeker

5. The method of claim 1, further comprising:
creating a perforation by advancing an instrument capable of making the perforation into the ethmoid infundibulum.

6. The method of claim 1, wherein the anteroposterior length of the ostium is 5 mm or greater.

7. The method of claim 1, wherein permanent deformation is achieved at only those sites chosen by the surgeon, and to a selected amount or degree.

8. The method of claim 1, wherein forces are applied to selected laminae whose displacement or deformation is desirable to restore mucociliary clearance, without manipulation of adjacent structures.

9. The method of claim 1, wherein a targeted laminae of each of an ostium is treated with a directed compression.

10. The method of claim 1, wherein maxillary and other sinuses are treated using adjacent opposing forces to either face of selected to generate a new convexity by fracturing a thin intervening bone.

11. The method of claim 1, wherein a risk of the superolateral and inferomedial walls becoming apposed for any MSO is reduced.

12. The method of claim 1, further comprising:
performing a targeted medial displacement of a medial wall of the maxillary sinus and uncinate using an uncinate medializing device.

13. The method of claim 1, further comprising:
remodeling a uncinate in a controlled fashion to a more medial for mucociliary flow position.

14. The method of claim 1, further comprising:
introducing a forward opening device anterograde via the keyhole instead of via a hiatus semilunaris.

15. The method of claim 1, wherein a distal flange is introduced into a MSO via a keyhole and a free end is directed inferiorly.

16. The method of claim 1, further comprising:
engaging mobile and fixed flanges of an apparatus; and
transfixing a medial wall of the maxillary sinus/lateral wall of a nose between the flanges as they are compressed into curviplanar shape flanges device surfaces.

17. The method of claim 1, further comprising:
displacing an inferomedial rim of a MSO from its superomedial rim.

18. The method of claim 1, further comprising:
locating a sphenoid ostium (SSO) with negligible anatomic variation on an anterolateral surface of the sphenoid rostrum.

19. The method of claim 1, further comprising:
providing directed anterolateral displacement of a posteromedial lamina of the superiormost cell of a agger nasi system; and
displacing an anterolateral wall of a frontal recess further anterolateral without manipulating a posteromedial wall of a recess.

20. The method of claim 1, further comprising:
providing drainage from a most of an anterior ethmoid by opening an EBO and HSS, and creating an anterior displacement of a medial margin of the EBO using adjacent opposing forces to lamina of an ethmoid bulla anteromedial to the EBO.

21. An apparatus for accessing and treating maxillary sinusitis, sphenoid sinusitis, and frontal sinusitis, comprising:
first and second distal flanges are coupled with or include first and second opposing jaws;
a shaft coupled to the first and second distal flanges; and
one or more handles coupled to the shaft.

22. The apparatus of claim 21, wherein one jaw is convex in cross-section, and a second jaw has a complementary concavity.

23. The apparatus of claim 21, wherein in response to application of a manual force to the handles, the first and second jaws are brought towards each other and transfix a sinus lamina between them

24. The apparatus of claim 23, wherein upon a release of a manual force the lamina retains a shape induced by an interlocking fit of the first and second jaws.

25. The method of claim 1, further comprising:
verifying dimensions of the MSO.

26. The apparatus of claim 21, wherein a distal assembly includes a distal mobile flange that is elliptical in cross section.

27. The apparatus of claim 21, wherein at least one of the first and second distal flanges has an angled distal assembly.

28. The apparatus of claim 21, wherein a second has pressure points on either side of and adjacent to a pressure point exerted by a first jaw.

29. The apparatus of claim 21, wherein the apparatus in operation provide for a fixed bone attachment.

30. The apparatus of claim 21, wherein the jaws in operation are brought together for transfixing without cutting or tearing an uncinate or its attachment.

31. The apparatus of claim 21, wherein the jaws in operation create a fracturing of a thin bone and an attachment suture.

32. The apparatus of claim 21, wherein the jaws in operation provide for a remodeling of an uncinate for a more medial for mucociliary flow position.

33. The apparatus of claim 21, wherein the apparatus is a forward-opening device that in operation is introduced anterograde via a keyhole 16.

34. The apparatus of claim 21, a single proximal flange is concave in surface with an opposing distal flange.

35. The apparatus of claim 34, wherein a radii of the an axes of a section of the distal flange is 1.5-4.5 mm

36. The apparatus of claim 21, wherein a gap of 0.5-1.5 mm is maintained between the flanges.

37. The apparatus of claim 21, wherein the flanges include an angulation.
